(19) United States
(12) Patent Application Publication
(10) Pub. No.: US 2011/0021929 A1
(43) Pub. Date: Jan. 27, 2011

(54) SYSTEMS AND METHODS FOR CONTINUOUS NON-INVASIVE BLOOD PRESSURE MONITORING

(75) Inventors: Rakesh Sethi, Vancouver (CA); James Watson, Dunfermline (GB); Paul Stanly Addison, Edinburgh (GB)

Correspondence Address: Nellcor Puritan Bennett LLC;ATTN: IP Legal, 6135 Gunbarrel Avenue, Boulder, CO, 80301, US

(73) Assignee: Nellcor Puritan Bennett Ireland, Mervue (IE)

(21) Appl. No.: 12/509,790

(22) Filed: Jul. 27, 2009

(51) Int. Cl. A61B 5/02

(52) U.S. Cl. 600/485

(57) ABSTRACT

Systems and methods are disclosed herein for continuous non-invasive blood pressure (CNIBP) monitoring. Multiple reference blood pressure values may be obtained using a calibration device. These multiple reference blood pressure values may be used as calibration points for determining a relationship between the blood pressure of a patient and photoplethysmograph (PPG) signals.

### SUMMARY

The present disclosure relates to signal processing and, more particularly, the present disclosure relates to systems and methods for continuous non-invasive blood pressure (CNIBP) monitoring. Multiple reference blood pressure values may be obtained using a calibration device. These multiple reference blood pressure values may be used as calibration points for determining a relationship between the blood pressure of a patient and photoplethysmograph (PPG) signals.

The disclosure relates to a blood pressure monitor, a method for monitoring blood pressure of a patient, and a computer-readable medium for use in monitoring blood pressure of a patient. The blood pressure monitor includes a signal generator for generating photoplethysmograph (PPG) signals from probes and/or sensors attached to a patient. The blood pressure monitor also includes a processor coupled to the signal generator. The processor is capable of determining multiple reference blood pressure values based at least in part on a calibration device coupled to the patient and the processor. The processor is also capable of updating a relationship between blood pressure of the patient and the PPG signals based at least in part on the multiple reference blood pressure values. The processor then calculates a blood pressure value based at least in part on the updated relationship. An output device is coupled to the processor.

In an embodiment, the processor is further capable of identifying points in the PPG signals after the multiple reference blood pressure values are obtained and determining a time difference between the points. The processor calculates the blood pressure value based at least in part on the time difference and the updated relationship. In an embodiment, the relationship is P=a+b·ln(T) or a mathematical equivalent thereof, where P is the blood pressure value, T is the time difference, and a and b are constants determined based at least in part on the multiple reference blood pressure values.

In an embodiment, the processor is farther capable of identifying a reference blood pressure value as an outlier. A new reference blood pressure value may be determined to verify the outlier.

In an embodiment, the processor is further capable of associating weighting factors with the multiple reference blood pressure values and updating the relationship between blood pressure of the patient and the PPG signals based at least in part on the multiple reference blood pressure values and the weighting factors.

In an embodiment, the processor is further capable of identifying a blood pressure event. After the blood pressure event, the processor is capable of determining further reference blood pressure values, resetting the relationship between blood pressure of the patient and the PPG signals, and updating the relationship between blood pressure of the patient and the PPG signals based at least in part on the further reference blood pressure values. The blood pressure event may be a change in arterial compliance. The blood pressure event may be a blood pressure change that exceeds a threshold.

### BRIEF DESCRIPTION OF THE DRAWINGS

The above and other features of the present disclosure, its nature and various advantages will be more apparent upon consideration of the following detailed description, taken in conjunction with the accompanying drawings in which:

FIG. 1 shows an illustrative pulse oximetry system in accordance with an embodiment;

FIG. 2 is a block diagram of the illustrative pulse oximetry system of FIG. 1 coupled to a patient in accordance with an embodiment;

FIG. 3 is a block diagram of an illustrative signal processing system in accordance with an embodiments;

FIG. 4 is a flow chart of an illustrative process for monitoring blood pressure using the pulse oximetry system of FIG. 1 in accordance with an embodiment; and

FIG. 5 is a flow chart of an illustrative process calibrating a blood pressure monitoring system operating according to the process of FIG. 4 in accordance with an embodiment.

### DETAILED DESCRIPTION

An oximeter is a medical device that may determine the oxygen saturation of the blood. One common type of oximeter is a pulse oximeter, which may indirectly measure the oxygen saturation of a patient's blood (as opposed to measuring oxygen saturation directly by analyzing a blood sample taken from the patient) and changes in blood volume in the skin. Ancillary to the blood oxygen saturation measurement, pulse oximeters may also be used to measure the pulse rate of the patient. Pulse oximeters typically measure and display various blood flow characteristics including, but not limited to, the oxygen saturation of hemoglobin in arterial blood.

An oximeter may include a light sensor that is placed at a site on a patient, typically a fingertip, toe, forehead or earlobe, or in the case of a neonate, across a foot. The oximeter may pass light using a light source through blood perfused tissue and photoelectrically sense the absorption of light in the tissue. In addition, locations which are not typically understood to be optimal for pulse oximetry serve as suitable sensor locations for the blood pressure monitoring processes described herein, including any location on the body that has a strong pulsatile arterial flow. For example, additional suitable sensor locations include, without limitation, the neck to monitor cartoid artery pulsatile flow, the wrist to monitor radial artery pulsatile flow, the inside of a patient's thigh to monitor femoral artery pulsatile flow, the ankle to monitor tibial artery pulsatile flow, and around or in front of the ear. Suitable sensors for these locations may include sensors for sensing absorbed light based on detecting reflected light. In all suitable locations, for example, the oximeter may measure the intensity of light that is received at the light sensor as a function of time. The oximeter may also include sensors at multiple locations. A signal representing light intensity versus time or a mathematical manipulation of this signal (e.g., a scaled version thereof, a log taken thereof a scaled version of a log taken thereof, etc.) may be referred to as the photoplethysmograph (PPG) signal. In addition, the term “PPG signal,” as used herein, may also refer to an absorption signal (i.e., representing the amount of light absorbed by the tissue) or any suitable mathematical manipulation thereof. The light intensity or the amount of light absorbed may then be used to calculate the amount of the blood constituent (e.g., oxyhemoglobin) being measured as well as the pulse rate and when each individual pulse occurs.

The light passed through the tissue is selected to be of one or more wavelengths that are absorbed by the blood in an amount representative of the amount of the blood constituent present in the blood. The amount of light passed through the tissue varies in accordance with the changing amount of blood constituent in the tissue and the related light absorption. Red and infrared wavelengths may be used because it has been observed that highly oxygenated blood will absorb relatively less red light and more infrared light than blood with a lower oxygen saturation. By comparing the intensities of two wavelengths at different points in the pulse cycle, it is possible to estimate the blood oxygen saturation of hemoglobin in arterial blood.

When the measured blood parameter is the oxygen saturation of hemoglobin, a convenient starting point assumes a saturation calculation based on Lambert-Beer's law. The following notation will be used herein:

I(λ,t)=Iₒ(λ)exp(−(sβₒ(λ)+(1−s)βᵣ(λ))l(t)) (1)

where:
λ=wavelength;
t=time;
I=intensity of light detected;
Iₒ=intensity of light transmitted;
s=oxygen saturation;
βₒ, βᵣ=empirically derived absorption coefficients; and
l(t)=a combination of concentration and path length from emitter to detector as a function of time.

The traditional approach measures light absorption at two wavelengths (e.g., red and infrared (IR)), and then calculates saturation by solving for the “ratio of ratios” as follows.
1. First, the natural logarithm of (1) is taken (“log” will be used to represent the natural logarithm) for IR and Red

log I=log Iₒ−(sβₒ+(1−s)βᵣ)l (2)
2. (2) is then differentiated with respect to time

$$\frac{d log I}{d t} = - ( s {β}_{o} + ( 1 - s ) {β}_{r} ) \frac{d l}{d t} \qquad ( 3 )$$

3. Red (3) is divided by IR (3)

$$\frac{d log I ( {λ}_{R} ) / d t}{d log I ( {λ}_{IR} ) / d t} = \frac{s {β}_{o} ( {λ}_{R} ) + ( 1 - s ) {β}_{r} ( {λ}_{R} )}{s {β}_{o} ( {λ}_{IR} ) + ( 1 - s ) {β}_{r} ( {λ}_{IR} )} \qquad ( 4 )$$

4 Solving for s

$$s = \frac{\frac{d log I ( {λ}_{IR} )}{d t} {β}_{r} ( {λ}_{R} ) - \frac{d log I ( {λ}_{R} )}{d t} {β}_{r} ( {λ}_{IR} )}{\begin{matrix}\frac{d log I ( {λ}_{R} )}{d t} ( {β}_{o} ( {λ}_{IR} ) - {β}_{r} ( {λ}_{IR} ) ) - \\ \frac{d log I ( {λ}_{IR} )}{d t} ( {β}_{o} ( {λ}_{R} ) - {β}_{r} ( {λ}_{R} ) )\end{matrix}}$$

Note in discrete time

$$\frac{d log I ( λ , t )}{d t} ≃ log I ( λ , {t}_{2} ) - log I ( λ , {t}_{1} )$$

### Using log A−log B=log A/B,

$$\frac{d log I ( λ , t )}{d t} ≃ log ( \frac{I ( {t}_{2} , λ )}{I ( {t}_{1} , λ )} )$$

So, (4) can be rewritten as

$$\frac{\frac{d log I ( {λ}_{R} )}{d t}}{\frac{d log I ( {λ}_{IR} )}{d t}} ≃ \frac{log ( \frac{I ( {t}_{1} , {λ}_{R} )}{I ( {t}_{2} , {λ}_{R} )} )}{log ( \frac{I ( {t}_{1} , {λ}_{IR} )}{I ( {t}_{2} , {λ}_{IR} )} )} = R \qquad ( 5 )$$

where R represents the “ratio of ratios.” Solving (4) for s using (5) gives

$$s = \frac{{β}_{r} ( {λ}_{R} ) - R {β}_{r} ( {λ}_{IR} )}{R ( {β}_{o} ( {λ}_{IR} ) - {β}_{r} ( {λ}_{IR} ) ) - {β}_{o} ( {λ}_{R} ) + {β}_{r} ( {λ}_{R} )} .$$

From (5), R can be calculated using two points (e.g., PPG maximum and minimum), or a family of points. One method using a family of points uses a modified version of (5). Using the relationship

$$\frac{d log I}{d t} = \frac{d I / d t}{I} \qquad ( 6 )$$

now (5) becomes

$$\begin{matrix}\frac{\frac{d log I ( {λ}_{R} )}{d t}}{\frac{d log I ( {λ}_{IR} )}{d t}} ≃ \frac{\frac{I ( {t}_{2} , {λ}_{R} ) - I ( {t}_{1} , {λ}_{R} )}{I ( {t}_{1} , {λ}_{R} )}}{\frac{I ( {t}_{2} , {λ}_{IR} ) - I ( {t}_{1} , {λ}_{IR} )}{I ( {t}_{1} , {λ}_{IR} )}} \\ = \frac{[ I ( {t}_{2} , {λ}_{R} ) - I ( {t}_{1} , {λ}_{R} ) ] I ( {t}_{1} , {λ}_{IR} )}{[ I ( {t}_{2} , {λ}_{IR} ) - I ( {t}_{1} , {λ}_{IR} ) ] I ( {t}_{1} , {λ}_{R} )} \\ = R\end{matrix} \qquad ( 7 )$$

which defines a cluster of points whose slope of y versus x will give R where

x(t)=[I(t₂,λ_{IR})−I(t₁,λ_{IR})]I(t₁,λ_{R})

y(t)=[I(t₂,λ_{R})−I(t₁,λ_{R})]I(t₁,λ_{IR})

y(t)=Rx(t) (8)

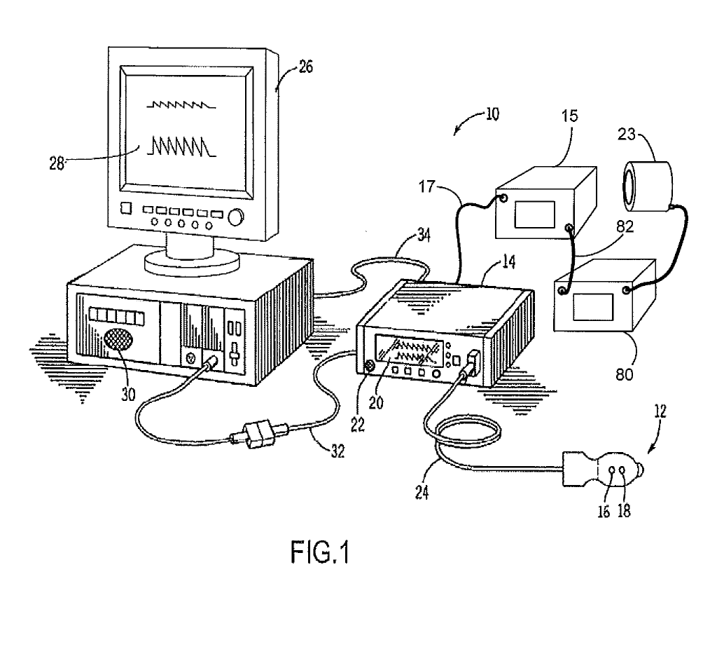

FIG. 1 is a perspective view of an embodiment of a pulse oximetry system 10. System 10 may include a sensor 12 and a pulse oximetry monitor 14. Sensor 12 may include an emitter 16 for emitting light at two or more wavelengths into a patient's tissue. A detector 18 may also be provided in sensor 12 for detecting the light originally from emitter 16 that emanates from the patient's tissue after passing through the tissue.

According to an embodiment and as will be described, system 10 may include a plurality of sensors forming a sensor array in lieu of single sensor 12. Each of the sensors of the sensor array may be a complementary metal oxide semiconductor (CMOS) sensor. Alternatively, each sensor of the array may be charged coupled device (CCD) sensor. In an embodiment, the sensor array may be made up of a combination of CMOS and CCD sensors. The CCD sensor may comprise a photoactive region and a transmission region for receiving and transmitting data whereas the CMOS sensor may be made up of an integrated circuit having an array of pixel sensors. Each pixel may have a photodetector and an active amplifier.

According to an embodiment, emitter 16 and detector 18 may be on opposite sides of a digit such as a finger or toe, in which case the light that is emanating from the tissue has passed completely through the digit. In an embodiment, emitter 16 and detector 18 may be arranged so that light from emitter 16 penetrates the tissue and is reflected by the tissue into detector 18, such as a sensor designed to obtain pulse oximetry data from a patient's forehead.

In an embodiment, the sensor or sensor array may be connected to and draw its power from monitor 14 as shown. In another embodiment, the sensor may be wirelessly connected to monitor 14 and include its own battery or similar power supply (not shown). Monitor 14 may be configured to calculate physiological parameters based at least in part on data received from sensor 12 relating to light emission and detection. In an alternative embodiment, the calculations may be performed on the monitoring device itself and the result of the oximetry reading may be passed to monitor 14. Further, monitor 14 may include a display 20 configured to display the physiological parameters or other information about the system. In the embodiment shown, monitor 14 may also include a speaker 22 to provide an audible sound that may be used in various other embodiments, such as for example, sounding an audible alarm in the event that a patient's physiological parameters are not within a predefined normal range. In an embodiment, the monitor 14 includes a blood pressure monitor 15. In alternative embodiments, the pulse oximetry system 10 includes a stand alone blood pressure monitor 15 in communication with the monitor 14 via a cable 17 or a wireless network link.

In an embodiment, sensor 12, or the sensor array, may be communicatively coupled to monitor 14 via a cable 24. However, in other embodiments, a wireless transmission device (not shown) or the like may be used instead of or in addition to cable 24.

In the illustrated embodiment, pulse oximetry system 10 may also include a multi-parameter patient monitor 26. The monitor may be cathode ray tube type, a flat panel display (as shown) such as a liquid crystal display (LCD) or a plasma display, or any other type of monitor now known or later developed. Multi-parameter patient monitor 26 may be configured to calculate physiological parameters and to provide a display 28 for information from monitor 14 and from other medical monitoring devices or systems (not shown). For example, multiparameter patient monitor 26 may be configured to display an estimate of a patient's blood oxygen saturation generated by pulse oximetry monitor 14 (referred to as an “SpO₂” measurement), pulse rate information from monitor 14 and blood pressure from blood pressure monitor 15 on display 28.

Monitor 14 may be communicatively coupled to multi-parameter patient monitor 26 via a cable 32 or 34 that is coupled to a sensor input port or a digital communications port, respectively and/or may communicate wirelessly (not shown). In addition, monitor 14 and/or multi-parameter patient monitor 26 may be coupled to a network to enable the sharing of information with servers or other workstations (not shown). Monitor 14 may be powered by a battery (not shown) or by a conventional power source such as a wall outlet.

Calibration device 80, which may be powered by monitor 14, a battery, or by a conventional power source such as a wall outlet, may include any suitable blood pressure calibration device. For example, calibration device 80 may take the form of any invasive or non-invasive blood pressure monitoring or measuring system used to generate reference blood pressure measurements for use in calibrating the CNIBP monitoring techniques described herein. Such calibration devices may include, for example, an aneroid or mercury sphygmomanometer and occluding cuff 23, a pressure sensor inserted directly into a suitable artery of a patient, an oscillometric device or any other device or mechanism used to sense, measure, determine, or derive a reference blood pressure measurement. In some embodiments, calibration device 80 may include a manual input device (not shown) used by an operator to manually input reference blood pressure measurements obtained from some other source (e.g., an external invasive or non-invasive blood pressure measurement system).

Calibration device 80 may also access reference blood pressure measurements stored in memory (e.g., RAM, ROM, or a storage device). For example, in some embodiments, calibration device 80 may access reference blood pressure measurements from a relational database stored within calibration device 80, monitor 14, or multi-parameter patient monitor 26. The reference blood pressure measurements generated or accessed by calibration device 80 may be updated in real-time, resulting in a continuous source of reference blood pressure measurements for use in continuous or periodic calibration. Alternatively, reference blood pressure measurements generated or accessed by calibration device 80 may be updated periodically, and calibration may be performed on the same periodic cycle. Preferably, the reference blood pressure measurements are generated when recalibration is triggered as described below. In the depicted embodiments, calibration device 80 is connected to monitor 14 or blood pressure monitor 15 via cable 82. In other embodiments, calibration device 80 may be a stand-alone device that may be in wireless communication with monitor 14. Reference blood pressure measurements may then be wirelessly transmitted to monitor 14 for use in calibration. In still other embodiments, calibration device 80 is completely integrated within monitor.

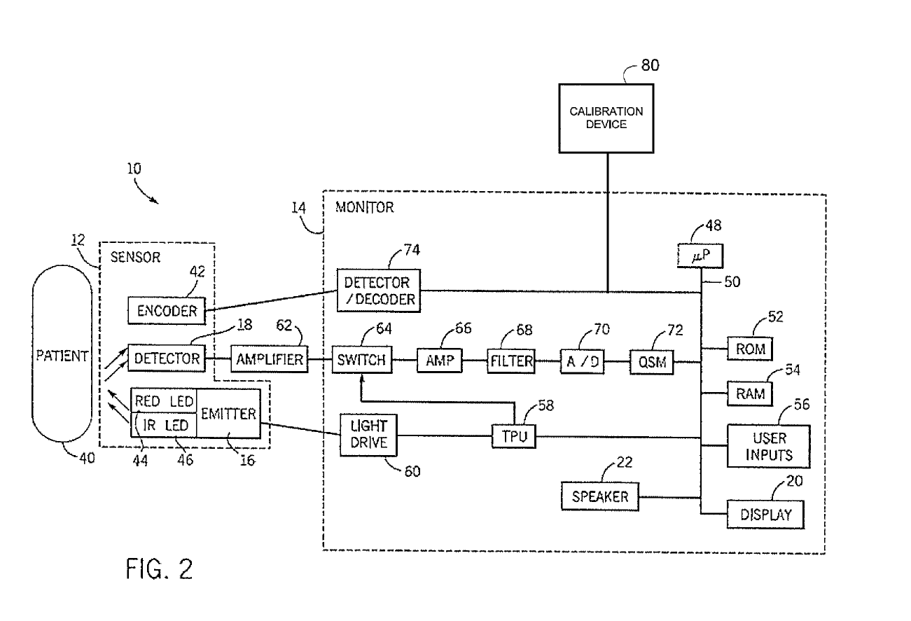

FIG. 2 is a block diagram of a pulse oximetry system, such as pulse oximetry system 10 of FIG. 1, which may be coupled to a patient 40 in accordance with an embodiment. Certain illustrative components of sensor 12 and monitor 14 are illustrated in FIG. 2. Sensor 12 may include emitter 16, detector 18, and encoder 42. In the embodiment shown, emitter 16 may be configured to emit at least two wavelengths of light (e.g., RED and IR) into a patient's tissue 40. Hence, emitter 16 may include a RED light emitting light source such as RED light emitting diode (LED) 44 and an IR light emitting light source such as IR LED 46 for emitting light into the patient's tissue 40 at the wavelengths used to calculate the patient's physiological parameters. In one embodiment, the RED wavelength may be between about 600 nm and about 700 nm, and the IR wavelength may be between about 800 nm and about 1000 nm. In embodiments where a sensor array is used in place of single sensor, each sensor may be configured to emit a single wavelength. For example, a first sensor emits only a RED light while a second only emits an IR light. In another example, the wavelengths of light used are selected based on the specific location of the sensor.

It will be understood that, as used herein, the term “light” may refer to energy produced by radiative sources and may include one or more of ultrasound, radio, microwave, millimeter wave, infrared, visible, ultraviolet, gamma ray or X-ray electromagnetic radiation. As used herein, light may also include any wavelength within the radio, microwave, infrared, visible, ultraviolet, or X-ray spectra, and that any suitable wavelength of electromagnetic radiation may be appropriate for use with the present techniques. Detector 18 may be chosen to be specifically sensitive to the chosen targeted energy spectrum of the emitter 16.

In an embodiment, detector 18 may be configured to detect the intensity of light at the RED and IR wavelengths. Alternatively, each sensor in the array may be configured to detect an intensity of a single wavelength. In operation, light may enter detector 18 after passing through the patient's tissue 40. Detector 18 may convert the intensity of the received light into an electrical signal. The light intensity is directly related to the absorbance and/or reflectance of light in the tissue 40. That is, when more light at a certain wavelength is absorbed or reflected, less light of that wavelength is received from the tissue by the detector 18. After converting the received light to an electrical signal, detector 18 may send the signal to monitor 14, where physiological parameters may be calculated based on the absorption of the RED and IR wavelengths in the patient's tissue 40.

In an embodiment, encoder 42 may contain information about sensor 12, such as what type of sensor it is (e.g., whether the sensor is intended for placement on a forehead or digit) and the wavelengths of light emitted by emitter 16. This information may be used by monitor 14 to select appropriate algorithms, lookup tables and/or calibration coefficients stored in monitor 14 for calculating the patient's physiological parameters.

Encoder 42 may contain information specific to patient 40, such as, for example, the patient's age, weight, and diagnosis. This information may allow monitor 14 to determine, for example, patient-specific threshold ranges in which the patient's physiological parameter measurements should fall and to enable or disable additional physiological parameter algorithms. Encoder 42 may, for instance, be a coded resistor which stores values corresponding to the type of sensor 12 or the type of each sensor in the sensor array, the wavelengths of light emitted by emitter 16 on each sensor of the sensor array, and/or the patient's characteristics. In another embodiment, encoder 42 may include a memory on which one or more of the following information may be stored for communication to monitor 14: the type of the sensor 12; the wavelengths of light emitted by emitter 16; the particular wavelength each sensor in the sensor array is monitoring; a signal threshold for each sensor in the sensor array; any other suitable information; or any combination thereof.

In an embodiment, signals from detector 18 and encoder 42 may be transmitted to monitor 14. In the embodiment shown, monitor 14 may include a general-purpose microprocessor 48 connected to an internal bus 50. Microprocessor 48 may be adapted to execute software, which may include an operating system and one or more applications, as part of performing the functions described herein. Also connected to bus 50 may be a read-only memory (ROM) 52, a random access memory (RAM) 54, user inputs 56, display 20, and speaker 22.

RAM 54 and ROM 52 are illustrated by way of example, and not limitation. Any suitable computer-readable media may be used in the system for data storage. Computer-readable media are capable of storing information that can be interpreted by microprocessor 48. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by components of the system.

In the embodiment shown, a time processing unit (TPU) 58 may provide timing control signals to a light drive circuitry 60, which may control when emitter 16 is illuminated and multiplexed timing for the RED LED 44 and the IR LED 46. TPU 58 may also control the gating-in of signals from detector 18 through an amplifier 62 and a switching circuit 64. These signals are sampled at the proper time, depending upon which light source is illuminated. The received signal from detector 18 may be passed through an amplifier 66, a low pass filter 68, and an analog-to-digital converter 70. The digital data may then be stored in a queued serial module (QSM) 72 (or buffer) for later downloading to RAM 54 as QSM 72 fills up. In one embodiment, there may be multiple separate parallel paths having amplifier 66, filter 68, and A/D converter 70 for multiple light wavelengths or spectra received.

In an embodiment, microprocessor 48 may determine the patient's physiological parameters, such as SpO₂ and pulse rate, using various algorithms and/or look-up tables based on the value of the received signals and/or data corresponding to the light received by detector 18. Signals corresponding to information about patient 40, and particularly about the intensity of light emanating from a patient's tissue over time, may be transmitted from encoder 42 to a decoder 74. These signals may include, for example, encoded information relating to patient characteristics. Decoder 74 may translate these signals to enable the microprocessor to determine the thresholds based on algorithms or look-up tables stored in ROM 52. User inputs 56 may be used to enter information about the patient, such as age, weight, height, diagnosis, medications, treatments, and so forth. In an embodiment, display 20 may exhibit a list of values which may generally apply to the patient, such as, for example, age ranges or medication families, which the user may select using user inputs 56.

The optical signal through the tissue can be degraded by noise, among other sources. One source of noise is ambient light that reaches the light detector. Another source of noise is electromagnetic coupling from other electronic instruments. Movement of the patient also introduces noise and affects the signal. For example, the contact between the detector and the skin, or the emitter and the skin, can be temporarily disrupted when movement causes either to move away from the skin. In addition, because blood is a fluid, it responds differently than the surrounding tissue to inertial effects, thus resulting in momentary changes in volume at the point to which the oximeter probe is attached.

Noise (e.g., from patient movement) can degrade a pulse oximetry signal relied upon by a physician, without the physician's awareness. This is especially true if the monitoring of the patient is remote, the motion is too small to be observed, or the doctor is watching the instrument or other parts of the patient, and not the sensor site. Processing pulse oximetry (i.e., PPG) signals may involve operations that reduce the amount of noise present in the signals or otherwise identify noise components in order to prevent them from affecting measurements of physiological parameters derived from the PPG signals.

It will be understood that the present disclosure is applicable to any suitable signals and that PPG signals are used merely for illustrative purposes. Those skilled in the art will recognize that the present disclosure has wide applicability to other signals including, but not limited to other biosignals (e.g., electrocardiogram, electroencephalogram, electrogastrogram, electromyogram, heart rate signals, pathological sounds, ultrasound, or any other suitable biosignal), dynamic signals, non-destructive testing signals, condition monitoring signals, fluid signals, geophysical signals, astronomical signals, electrical signals, financial signals including financial indices, sound and speech signals, chemical signals, meteorological signals including climate signals, and/or any other suitable signal, and/or any combination thereof.

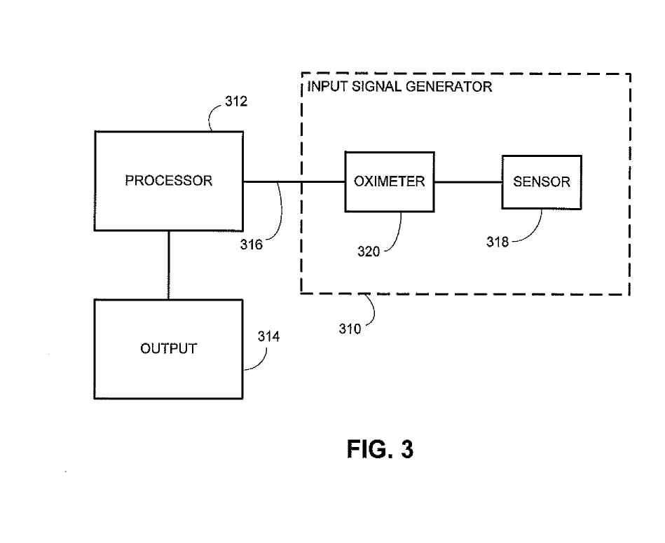

FIG. 3 is an illustrative signal processing system in accordance with an embodiment. In this embodiment, input signal generator 310 generates an input signal 316. As illustrated, input signal generator 310 may include oximeter 320 coupled to sensor 318, which may provide as input signal 316, a PPG signal. It will be understood that input signal generator 310 may include any suitable signal source, signal generating data, signal generating equipment, or any combination thereof to produce signal 316. Signal 316 may be any suitable signal or signals, such as, for example, biosignals (e.g., electrocardiogram, electroencephalogram, electrogastrogram, electromyogram, heart rate signals, pathological sounds, ultrasound, or any other suitable biosignal), dynamic signals, nondestructive testing signals, condition monitoring signals, fluid signals, geophysical signals, astronomical signals, electrical signals, financial signals including financial indices, sound and speech signals, chemical signals, meteorological signals including climate signals, and/or any other suitable signal, and/or any combination thereof.

In this embodiment, signal 316 may be coupled to processor 312. Processor 312 may be any suitable software, firmware, and/or hardware, and/or combinations thereof for processing signal 316. For example, processor 312 may include one or more hardware processors (e.g., integrated circuits), one or more software modules, computer-readable media such as memory, firmware, or any combination thereof Processor 312 may, for example, be a computer or may be one or more chips (i.e., integrated circuits) Processor 312 may perform the calculations associated with the signal processing of the present disclosure as well as the calculations associated with any calibration of the signal processing system. Processor 312 may perform any suitable signal processing of signal 316 to filter signal 316, such as any suitable band-pass filtering, adaptive filtering, closed-loop filtering, and/or any other suitable filtering, and/or any combination thereof.

Processor 312 may be coupled to one or more memory devices (not shown) or incorporate one or more memory devices such as any suitable volatile memory device (e.g., RAM, registers, etc.), non-volatile memory device (e.g., ROM, EPROM, magnetic storage device, optical storage device, flash memory, etc.), or both. The memory may be used by processor 312 to, for example, store data corresponding to store blood pressure monitoring data, including current blood pressure calibration values, blood pressure monitoring calibration thresholds, and patient blood pressure history.

Processor 312 may be coupled to output 314. Output 314 may be any suitable output device such as, for example, one or more medical devices (e.g., a medical monitor that displays various physiological parameters, a medical alarm, or any other suitable medical device that either displays physiological parameters or uses the output of processor 312 as an input), one or more display devices (e.g., monitor, PDA, mobile phone, any other suitable display device, or any combination thereof), one or more audio devices, one or more memory devices (e.g., hard disk drive, flash memory, RAM, optical disk, any other suitable memory device, or any combination thereof), one or more printing devices, any other suitable output device, or any combination thereof.

It will be understood that system 300 may be incorporated into system 10 (FIGS. 1 and 2) in which, for example, input signal generator 310 may be implemented as parts of sensor 12 and monitor 14 and processor 312 may be implemented as part of monitor 14.

Pulse oximeters, in addition to providing other information, can be utilized for continuous non-invasive blood pressure monitoring. As described in U.S. Pat. No. 6,599,251, the entirety of which is incorporated herein by reference, PPG and other pulse signals obtained from multiple probes can be processed to calculate the blood pressure of a patient. In particular, blood pressure measurements may be derived based on a comparison of time differences between certain components of the pulse signals detected at each of the respective probes. As described in U.S. Patent Application No. ______ (Attorney Docket No. H-RM-01205 (COV-11)), entitled “Systems and Methods For Non-Invasive Blood Pressure Monitoring,” and filed on Sep. 30, 2008, the entirety of which is incorporated herein by reference, blood pressure can also be derived by processing time delays detected within a single PPG or pulse signal obtained from a single pulse oximeter probe. In addition, as described in U.S. patent application Ser. No. ______ (Attorney Docket No. H-RM-01206 (COV-13)), entitled “Systems and Methods For Non-Invasive Continuous Blood Pressure Determination,” and filed on Sep. 30, 2008, the entirety of which is incorporated herein by reference, blood pressure may also be obtained by calculating the area under certain portions of a pulse signal. Finally, as described in U.S. patent application Ser. No. ______ (Attorney Docket No. H-RM-01233 (COV-38)), entitled “Systems and Methods For Maintaining Blood Pressure Monitor Calibration,” and filed on Sep. 30, 2008, the entirety of which is incorporated herein by reference, a blood pressure monitoring device may be recalibrated in response to arterial compliance changes.

One benefit of monitoring blood pressure based on PPG signals is that such signals can be obtained in a non-invasive fashion. To continuously monitor blood pressure using a conventional sphygmomanometer, a cuff is repeatedly inflated around a patient's appendage, applying significant pressure. Such repeated pressure can result at a minimum in patient discomfort and potentially in serious injury. In contrast, continuous blood pressure monitoring based on a pulse signal may be achieved merely by placing one or more pulse oximetry probes on appendages and/or other parts of a patient's body.

Some CNIBP monitoring techniques utilize two probes or sensors positioned at two different locations on a subject's body. The elapsed time, T, between the arrivals of corresponding points of a pulse signal at the two locations may then be determined using signals obtained by the two probes or sensors. The estimated blood pressure, P, may then be related to the elapsed time, T, by

P=a+b.ln(T) (9)

where a and b are constants that may be dependent upon the nature of the subject and the nature of the signal detecting devices. Other suitable equations using an elapsed time between corresponding points of a pulse signal may also be used to derive an estimated blood pressure measurement.

In an embodiment, multi-parameter equation (9) may include a non-linear function which is monotonically decreasing and concave upward in a manner specified by the constant parameters.

Equation (9) may be used to calculate the estimated blood pressure from the time difference, T, between corresponding points of a pulse signal received by two sensors or probes attached to two different locations of a subject. The value used for the time difference, T, in equation (9) (or in any other blood pressure equation using an elapsed time value between corresponding points of a pulse signal) may also be derived from a signal obtained from a single sensor or probe. In some embodiments, the signal obtained from the single sensor or probe may take the form of a PPG signal obtained, for example, from a CNIBP monitoring system or pulse oximeter. The time difference, T, may also be referred to as the differential pulse transit time (DPTT).

In an embodiment, constants a and b in equation (9) above may be determined by performing a calibration. The calibration may involve taking a reference blood pressure reading to obtain a reference blood pressure P₀, measuring the elapsed time T₀ corresponding to the reference blood pressure, and then determining values for both of the constants a and b from the reference blood pressure and elapsed time measurement. Calibration may be performed at any suitable time (e.g., once initially after monitoring begins) or on any suitable schedule (e.g., a periodic or event-driven schedule).

In an embodiment, the calibration may include performing calculations mathematically equivalent to

$$\begin{matrix}a = {c}_{1} + \frac{{c}_{2} ( {P}_{0} - {c}_{1} )}{ln ( {T}_{0} ) + {c}_{2}} \\ and & ( 10 ) \\ b = \frac{{P}_{0} - {c}_{1}}{ln ( {T}_{0} ) + {c}_{2}} & ( 11 )\end{matrix}$$

to obtain values for the constants a and b, where c₁ and c₂ are parameters that may be determined, for example, based on empirical data.

In an embodiment, the calibration may include performing calculations mathematically equivalent to

a=P₀−(c₃T₀+c₄)ln(T₀) (12)

and

b=c₃T₀+c₄ (13)

where a and b are first and second parameters and c₃ and c₄ are parameters that may be determined, for example, based on empirical data.

Parameters c₁, c₂, c₃, and c₄ may be predetermined constants empirically derived based on experimental data from a number of different patients. A single reference blood pressure reading from a patient, including reference blood pressure P₀ and elapsed time T₀ from one or more signals corresponding to that reference blood pressure, may be combined with this inter-patient data to calculate the blood pressure of a patient. The values of P₀ and T₀ may be referred to herein as a calibration point. According to this example, a single calibration point may be used

with the predetermined constant parameters to determine values of constants a and b for the patient (e.g., using equations (10) and (11) or (12) and (13)). Then blood pressure for the patient may then be calculated using equation (9). For this calibration to remain accurate, certain physiological characteristics of the patient should remain relatively constant. Significant changes in these characteristics may result in less accurate blood pressure readings, making recalibration desirable. Recalibration may be performed by collecting a new calibration point a recalculating the constants a and b used in equation (9). Calibration and recalibration may be performed using calibration device 80 (FIG. 1).

This single calibration point blood pressure estimation technique may require frequent recalibration to maintain the accuracy of the blood pressure estimations. For example, the single calibration point technique may provide less accurate results after a large change in blood pressure (e.g., 20 mmHg to 30 mmHg from the calibration point). As another example, the single calibration point technique may provide less accurate results after a change in the compliance or alternatively, the elasticity, of the arteries of the patient. Each recalibration will result in the calculation of new values for the constants used to estimate blood pressure, as described above. Processes and algorithms for initiating recalibration are described in the patent and patent applications incorporated by reference above.

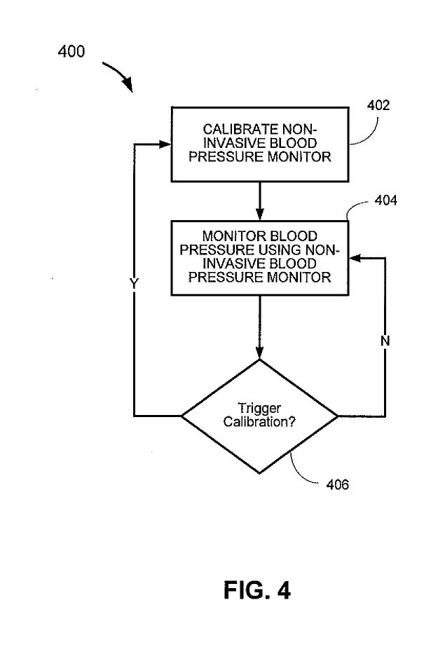

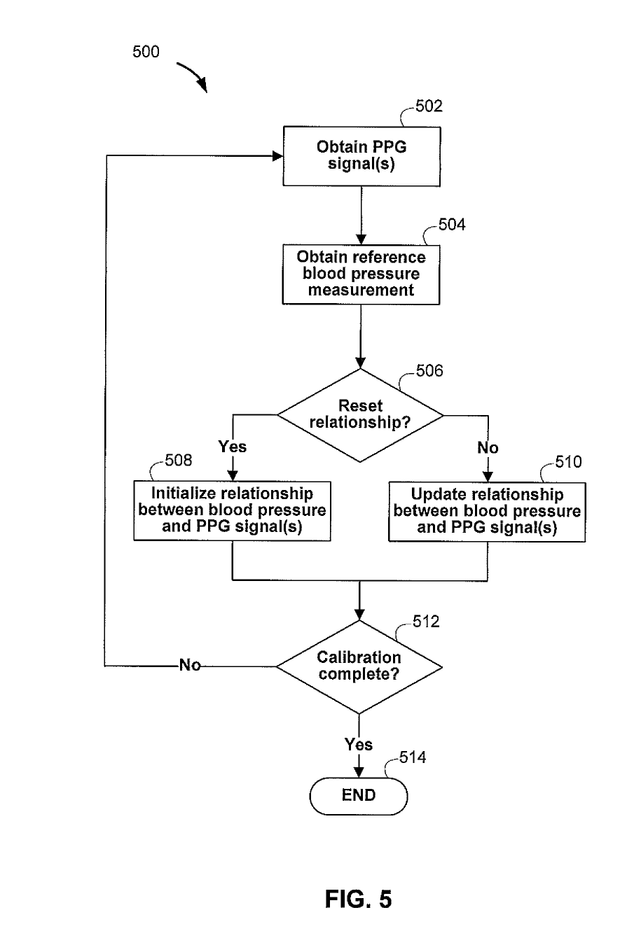

In an embodiment, multiple calibration points may be used to determine the relationship between a patient's blood pressure and one or more PPG signals. Using multiple calibration points to calculate this relationship may preferably provide a more accurate estimation of a patient's blood pressure than using the single calibration point described above. This relationship may be liner or non-linear and may be extrapolated and/or interpolated to define the relationship over the range of the collected recalibration data. For example, the multiple calibration points may be used to determine values for parameters c₁ and c₂ or c₃ and c₄, described above. These determined values will be based on information about the patient (intra-patient data) instead of information that came from multiple patients (intra-patient data) and may provide more accurate blood pressure estimation for the patient. As another example, the multiple calibration points may be used to determine values for parameters a and b, described above. Instead of calculating values of parameters a and b using a single calibration point and predetermined constants, values for parameters a and b may be empirically derived from the values of the multiple calibration points. As yet another example, the multiple calibration points may be used directly to determine the relationship between blood pressure and PPG signals. Instead of using a predefined relationship (e.g., the relationship defined by equation (9)), a relationship may be directly determined from the calibration points, for example, other linear or nonlinear functions may be fitted to the calibration points. In a further embodiment the linear or nonlinear function may be chosen with consideration to values of the calibration points collected. For example if calibration points for many varying blood pressures have been collected then a multi order polynomial fit of that data may be used to model the relationship. However, if only calibration points of constant pressure values have been collected then a logarithmic curve of the type of equation (9) and based on historical data may be used. Those skilled in the art will appreciate that the formula chosen to model the relationship may therefore change as additional calibration points are acquired. Processes for using multiple calibration points to determine the relationship between a patient's blood pressure and PPG signals are described in more detail below with reference FIG. 4 and FIG. 5.

FIG. 4 is a flow chart of an illustrative process 400 for monitoring blood pressure using the pulse oximetry system 10 of FIG. 1 in accordance with an embodiment. At step 402, a non-invasive blood pressure monitor 15 incorporated into or in communication with the pulse oximetry system 10 is calibrated using multiple calibration points. One illustrative process for calibrating the blood pressure monitor 15 using multiple calibration points is described further below in relation to FIG. 5. After calibration, at step 404, the non-invasive blood pressure monitor 15 monitors the blood pressure of the patient for which it was calibrated using pulse oximetry data collected by the pulse oximetry system 10. Suitable methods and systems for such monitoring, include, without limitation, those described in the patent and patent applications incorporated by reference above. At step 404, blood pressure monitor 15 determines whether to trigger recalibration. Recalibration may be performed at any suitable time. For example, blood pressure monitor 15 may trigger recalibration periodically (e.g, every 5 to 10 minutes). As another example, blood pressure monitor 15 may trigger recalibration based on changes in the monitored physiological characteristics of the patient. Blood pressure monitor 15 may trigger recalibration in response to detecting a change in the arterial compliance of the patient or in response to a threshold change in the blood pressure of the patient. As another example, blood pressure monitor 15 may trigger recalibration in response to the request device user. If recalibration is triggered, at step 402 blood pressure monitor 15 is calibrated, for example using calibration device 80. Otherwise, at step 404, the blood pressure monitor 15 continues to monitor blood pressure of the patient. Recalibration may be performed regularly in order to obtain enough calibration points to improve the accuracy of the blood pressure monitoring system.

FIG. 5 is a flow chart of an illustrative process 500 for calibrating a blood pressure monitoring system operating according to the method of FIG. 4 in accordance with an embodiment. Process 500 begins with blood pressure monitor 15 obtaining a one or more pulse signals, such as a PPG signal from pulse oximetry system 10 at step 502. At step 504, blood pressure monitor 15 obtains a reference blood pressure measurement, for example, using calibration device 80. For example, calibration device 80 may obtain a reference blood pressure measurement using any invasive or non-invasive blood pressure monitoring or measuring system. Such calibration devices may include, for example, an aneroid or mercury sphygmomanometer and occluding cuff 23, a pressure sensor inserted directly into a suitable artery of a patient, an oscillometric device or any other device or mechanism used to sense, measure, determine, or derive a reference blood pressure measurement. In some embodiments, calibration device 80 may include a manual input device (not shown) used by an operator to manually input reference blood pressure measurements obtained from some other source (e.g., an external invasive or non-invasive blood pressure measurement system). At step 506, blood pressure monitor 15 determines whether the relationship between a patient's blood pressure and the PPG signal(s) should be or has been reset. For example, the relationship may be reset: 1) initially after device or monitoring initialization; 2) after a threshold change in monitored physiological characteristics of the patient (e.g., arterial compliance); 3) periodically (e.g., once a day); 4) at the request of the device user; or 5) at any combination of the aforementioned times.

After the relationship is reset, new calibration points may collected and the previous calibration points may be discarded. If there is a significant change in the values of the new calibration points obtained (as compared to previous calibration points) and/or if there are significant physiological changes in the patient (e.g., changes in arterial compliance or blood pressure), the relationship may be reset in order to determine a new relationship based on the current data. Similarly, the relationship may be reset on a periodic basis (e.g., every day) in order to refresh the relationship with current data. Additionally or alternatively, the relationship may be reset if the accuracy of the calculated blood pressure falls below a given threshold.

If the relationship between a patient's blood pressure and the PPG signal(s) should be or has been reset, at step 508, the relationship between blood pressure and the PPG signal(s) is initialized, for example, using calibration device 80. The relationship may be initialized using multiple calibration points to determine the relationship between a patient's blood pressure and the DPTT of one or more PPG signals. These multiple calibration points may include a calibration point determined based on the PPG signal(s) obtained at step 502 and the reference blood pressure measurement obtained at step 504 and may include additional calibration points based on additional PPG signals and blood pressure measurements.

In an embodiment, initialization may only require a single calibration point. As described above, the relationship between a patient's blood pressure and PPG signals may be calculated from equation (9) based on a single calibration point from the patient and predetermined constants from empirical data obtained from multiple patients. In this embodiment, the relationship may be initialized using a single calibration point and may be updated (at step 510) as new calibration points arc obtained. In this manner historical, inter-patient data may be used to initialize the relationship, but as new calibration points are collected the relationship may be refined using the patient specific data. In an embodiment, during initialization multiple calibration points may be collected and may be used to initialize the relationship. For example, the relationship may be initialized based on three or four calibration points. These multiple calibration points may be used independently or in combination with historical, inter-patient data.

If the relationship between a patient's blood pressure and the PPG signal(s) is not reset at step 508, the relationship between blood pressure and the PPG signal(s) is updated with a calibration point based on the PPG signal(s) obtained at step 502 and the reference blood pressure measurement obtained at step 504. This calibration point may be added to previously obtained calibration points to refine the relationship between a patient's blood pressure and the PPG signal(s). For example, this relationship may be updated by triggering recalibration of blood pressure monitor 15 with a new calibration point on a periodic basis (e.g., every 5-10 minutes). In an embodiment, every calibration point obtained may be used to refine the relationship between a patient's blood pressure and the PPG signal(s). In this manner, the relationship may be refined based on a relatively large data set. This data set may yield a blood pressure, PPG relationship that may be accurate across a wider set of circumstances than a relationship based on a single calibration point.

In an embodiment, the multiple calibration points used to calculate this relationship may be weighted differently. For example, more recent calibration points may be given more weight than older calibration points. As another example, calibration points that are deemed to be outliers from the determined relationship may be given less weight or even excluded entirely. Furthermore, if a calibration point is deemed to be an outlier a new calibration measurement may be triggered to verify if that previous calibration point was an outlier or merely represents a significant change in the obtained data.

At step 512 it is determined whether calibration is complete. If calibration is complete, process 500 ends at step 514. If calibration is not complete, additional calibration points may be obtained by repeating process 500.

The foregoing is merely illustrative of the principles of this disclosure and various modifications can be made by those skilled in the art without departing from the scope and spirit of the disclosure. The above described embodiments are presented for purposes of illustration and not of limitation. The present disclosure also can take many forms other than those explicitly described herein. Accordingly, it is emphasized that the disclosure is not limited to the explicitly disclosed methods, systems, and apparatuses, but is intended to include variations to and modifications thereof which are within the spirit of the following claims.

What is claimed is:

1. A system for monitoring blood pressure of a patient, the system comprising:
a signal generator for generating at least two PPG signals from at least two respective sensors attached to the patient;
a processor coupled to the signal generator, wherein the processor is capable of:
determining at least two reference blood pressure values based at least in part on a calibration device coupled to the patient and to the processor, and
updating a relationship between blood pressure of the patient and the at least two PPG signals based at least in part on the at least two reference blood pressure values,
calculating a blood pressure value based at least in part on the updated relationship; and
an output device coupled to the processor.

2. The system of claim 1, wherein the processor is further capable of:
identifying at least two points in the at least two PPG signals, wherein the at least two points occur after the reference blood pressure value is obtained;
determining a time difference between the at least two points; and
calculating the blood pressure value based at least in part on the time difference and the updated relationship.

3. The system of claim 2, wherein the relationship is
P=a+b·ln(T)
or a mathematical equivalent thereof, wherein P is the blood pressure value, T is the time difference, and a and b are constants determined based at least in part on the at least two reference blood pressure values.

4. The system of claim 1, wherein the processor is further capable of identifying a reference blood pressure value as an outlier.

5. The system of claim 4, wherein the processor is further capable of determining a new reference blood pressure value to verify the outlier.

6. The system of claim 1, wherein the processor is further capable of:
associating weighting factors with the at least two reference blood pressure values; and
updating the relationship based at least in part on the at least two reference blood pressure values and the weighting factors.

7. The system of claim 1, wherein the processor is further capable of:
identifying a blood pressure event;
determining at least two further reference blood pressure values after the blood pressure event occurs;
resetting the relationship between blood pressure of the patient and the at least two PPG signals; and
updating the relationship based at least in part on the at least two further reference blood pressure values.

8. The system of claim 7, wherein the blood pressure event is a change in vascular compliance.

9. The system of claim 7, wherein the blood pressure event is a blood pressure change that exceeds a threshold stored in the processor.

10. A method for monitoring blood pressure of a patient, the method comprising:
determining using a processor at least two reference blood pressure values based at least in part on a calibration device coupled to the patient and to the processor;
obtaining at least two PPG signals from at least two respective sensors attached to the patient;
updating a relationship between blood pressure of the patient and the at least two PPG signals based at least in part on the at least two reference blood pressure values; and
calculating a blood pressure value based at least in part on the updated relationship.

11. The method of claim 10, further comprising:
identifying at least two points in the at least two PPG signals, wherein the at least two points occur after the at least two reference blood pressure values are obtained;
determining a time difference between the at least two points; and
calculating the blood pressure value based at least in part on the time difference and the updated relationship.

12. The method of claim 10, wherein the relationship is
P=a+b·ln(T)
or a mathematical equivalent thereof, where P is the blood pressure value, T is the time difference, and a and b are constants determined based at least in part on the at least two reference blood pressure values.

13. The method of claim 10, further comprising identifying a blood pressure value as an outlier.

14. The method of claim 13, further comprising determining a new reference blood pressure value to verify the outlier.

15. The method of claim 10, further comprising:
associating weighting factors with the at least two reference blood pressure values; and
updating the relationship based at least in part on the at least two reference blood pressure values and the weighting factors.

16. The method of claim 10, further comprising:
identifying a blood pressure event;
determining at least two further reference blood pressure values after the blood pressure event occurs;
resetting the relationship between blood pressure of the patient and the at least two PPG signals; and
updating the relationship based at least in part on the at least two further reference blood pressure values.

17. The method of claim 16, wherein the blood pressure event is a change in vascular compliance.

18. The method of claim 16, wherein the blood pressure event is a blood pressure change that exceeds a threshold stored in the processor.

19. A computer-readable medium for use in monitoring blood pressure of a patient, the computer-readable medium having computer program instructions recorded thereon for:
determining at least two reference blood pressure values based at least in part on a calibration device coupled to the patient;
obtaining at least two PPG signals from at least two respective sensors attached to the patient;
updating a relationship between blood pressure of the patient and the at least two PPG signals based at least in part on the at least two reference blood pressure values; and
calculating a blood pressure value based at least in part on the updated relationship.
